(19) United States
(12) Patent Application Publication
(10) Pub. No.: US 2006/0217746 A1
(43) Pub. Date: Sep. 28, 2006

(54) Renal perfusion clamp

(76) Inventor: Arthur M.M. Krolman, Boston, MA (US)

Correspondence Address: BURNS & LEVINSON LLP, 1030 15TH STREET NW, SUITE 300, WASHINGTON, DC, 20005-1501, US

(21) Appl. No.: 11/089,295

(22) Filed: Mar. 24, 2005

(51) Int. Cl. A61B 17/08

(52) U.S. Cl. 606/151

(57) ABSTRACT

A single-hand operated renal perfusion clamp for the adjustable control of aorta patch compression directly at the clamp's distal point. The clamp features an offset hinge and an adjustable tension control J-hook for greater even sealing pressure across aorta patch.

### FIELD OF THE INVENTION

The present application relates generally to surgical clamping devices and, more particularly, to a renal perfusion clamp.

### BACKGROUND OF THE INVENTION

The renal perfusion clamp improves an organ transplant team's ability to perform renal perfusion on donor kidneys. When the donor kidney is not connected to the body's blood supply, it is typical for a solution of perfusate to be pumped into the renal aorta to ensure that a semblance of normal kidney function occurs immediately after the transplant operation has been completed. The perfusate supplies the kidney cells with nutrients and protects them with antibiotics, antioxidants, anti-inflammatory drugs, calcium channel blockers, and immunosuppressive agents. A clamp is coupled to a cannula through which the perfusate flows. The renal perfusion clamp of the present invention provides improved accuracy of clamp location on aorta patch and improved sealing of perfusion cannula to reduce the leakage of perfusate, and improved perfusate flow.

The perfusionist has alternate methods of cannulation which may be used according to his/her discretion, but with significant disadvantages. One is to insert a plastic cannula into the artery and suture it into place. This process causes the arterial tissue from the suture back to the aortic cuff to necrose, which significantly shortens the artery length usable during the transplant operation. The other alternative is to suture more aortic tissue (if available) around the distal end of the artery in order to create a sufficient amount of tissue to effectively use the clamp. This procedure significantly increases the amount of time that the organ must remain on ice, which detrimentally affects the preservation of the organ.

In order to couple the perfusion cannula to the renal aorta, prior art clamps have been developed. A prior art clamp is illustrated in FIG. 1-3 and indicated generally at 10. The clamp 10 is manufactured by Pilling Surgical of Horsham, Pa. The clamp 10 includes two longitudinal members 12 and 14 which pivot about a pin 16. The proximal end of longitudinal member 12 includes a handle 18, while the proximal end of the member 14 includes a handle 20. The distal end of the member 12 includes a clamp head 24, while the distal end of the member 14 includes a clamp head 26. Clamp head 26 includes a nipple 28 attached thereto. Movement of the handles 18 and 20 toward each other forces the members 12 and 14 to pivot about the pin 16. This forces the clamp heads 24 and 26 away from each other. A spring 22 is positioned between the handles 18 and 20 in order to bias the handles apart. This serves to force clamp heads 24 and 26 together.

In FIG. 2, it can be seen that the proximal ends of members 12 and 14 comprise elongated and narrow handles. FIG. 3 illustrates the prior art clamp 10 in use. A catheter 40 is attached in order to provide perfusion of liquid through the lumen 32 and into the renal aorta 34. The spring 22 of the prior art clamp 10 exhibits several problems which increase the difficulty in using the clamp 10 during perfusion of donor kidney 36. The spring 22 does not provide proper sealing of the aortic patch 38 between the clamp heads. This results in unnecessary leakage of perfusion fluid. Long-term use of the prior art clamp 10 results in wear on spring 22, which further reduces the efficiency of the sealing mechanism of the clamp 10. The position of the spring 22 on the proximal end of the clamp minimizes the sealing force that is needed to keep the clamp heads 24 and 26 closed and the aortic patch secure in the opposite end of the clamp.

Finally, the spring 22 prevents the handles 18 and 20 to be compressed closely. This results in a narrow maximum opening between clamp heads 24 and 26, leaving a small space for the perfusionist to insert the aorta patch into the clamp.

The Westcott patent, U.S. Pat. No. 5,728,115, is an attempt to address some of the disadvantages with prior art clamp 10. However, there are several features of the Westcott perfusion clamp that present a new set of problems that the present invention is designed to solve. The clamp contains two longitudinal arms with a maximum opening angle of 180 degrees which must be brought together by perfusionist. A clamping bar is inserted and screwed through the arms to maintain the arms in a clamping position. The perfusionist must then fix the position of the clamping bar through the use of a thumbwheel. This causes the perfusionist to move his or her hands around and under the clamp a number of times in order to obtain maximum tightness of the clamping mechanism. This increases the risk of loosening the aortic patch from its desired position as the tightness of the clamp is adjusted.

The Westcott clamp is also supplied with assorted clamp heads that can be screwed on to fit various aorta patch sizes. In conjunction with the releasable components of the first and second arms, the use of multiple clamp heads presents a large number of loose parts parts that a perfusionist could misplace or fail to screw in place properly. Achieving desirable sterility is also more complicated as the removable parts of the Westcott invention provide more spaces and crevices from which bacteria and other microorganisms may not be sufficiently eliminated.

Thus, the prior art clamps aforementioned have shortcomings, including those related to proper sealing and one-handed operation. Concerning proper sealing, the prior art clamps offer a clamping force towards the proximal end of the clamp. This may cause perfusate leakage in the common situation where calcification of the donor's aorta wall present one or more hard bumps of calcium deposits preventing complete closure of the clamp's distal tip. Adjustable clamping force transmitted through the clamp arms from a location towards the proximal end of the clamp may be insufficient to completely compress such bumps to close the clamp completely and create an effective seal.

There is therefore a need for a perfusion clamp which overcomes the problems inherent in the prior art design. The present invention is directed toward meeting this need.

It is an object of the present invention to provide a one-hand operated renal perfusion clamp which allows for adjustable control of aorta patch compression directly at the clamp's distal point.

It is another object of the present invention to provide a more even seal of the aorta patch over otherwise unmanageable calcium deposits to reduce the risk of perfusate leakage and flow reduction inside the perfused donor kidney.

Another object of the present invention is to provide a wide degree of opening between the clamp heads to allow for insertion of forceps to pull aorta patch into the clamp.

Another object of the present invention is to provide increased stability of the clamp within the grip of the perfusionist's hand.

It is yet another object of the invention to reduce the cannula length without increasing the risk of slippage of the perfusion pump silicone tubing, which will provide a plurality of beneficial results.

An additional object of the present invention is to prevent accidental misplacement of the adjustable control mechanism through the use of capped ends.

Still another object of the invention is to provide a perfusion clamp which may be manufactured in various sizes to address the variable size and thickness of the donor kidney's aortic patch.

### SUMMARY OF THE INVENTION

The disclosed invention addresses all of the previously mentioned shortfalls in providing maximum sealing of the aortic patch and reduced perfusate leakage during the removal of the donor kidney.

The invention may be manufactured in various sizes to achieve maximum operability in patients having various aortic patch sizes and thicknesses.

The invention relates to a perfusion clamp comprising a first longitudinal arm; a second longitudinal arm; a spring mechanism, wherein a first end of a hinge is coupled to a proximal end of the first arm and a second end of said hinge is coupled to a proximal end of the second arm, and wherein at least one of the first and second ends pivots at its coupling; a first clamp head coupled to a distal end of the first arm; a second clamp head coupled to a distal end of the second arm, said second clamp head including a perfusion lumen extending therethrough; and a J-hook adjustable clamping mechanism coupled to the first and second arms and operative to releasably maintain the first and second clamp heads in clamping engagement with adjustable force.

### BRIEF DESCRIPTION OF THE DRAWINGS

FIG. 1 is a side elevational view of a prior art perfusion clamp.

FIG. 2 is a top plan view of the prior art perfusion clamp of FIG. 1.

FIG. 3 is a perspective view of the prior art perfusion clamp of FIG. 1 in use with a donor kidney.

FIG. 4 is a side view of an example of the perfusion clamp of the present invention.

FIG. 5 is a top plan view of an example of the perfusion clamp of the present invention.

FIG. 6 is a side view of an example of the perfusion clamp of the present invention in an open position.

FIG. 7 is a side view of the preferred embodiment perfusion clamp of the present invention in an open position in which the fulcrum is offset.

FIG. 8 is a side view of the preferred embodiment perfusion clamp of the present invention in a closed position in which the fulcrum is offset and in use with a donor kidney.

FIG. 9 is a view of a hinge and a spring mechanism in accordance with the present invention.

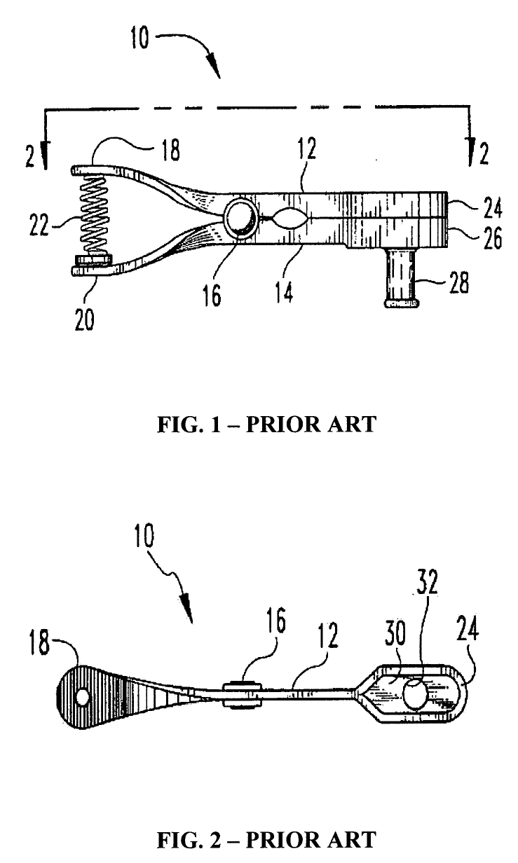

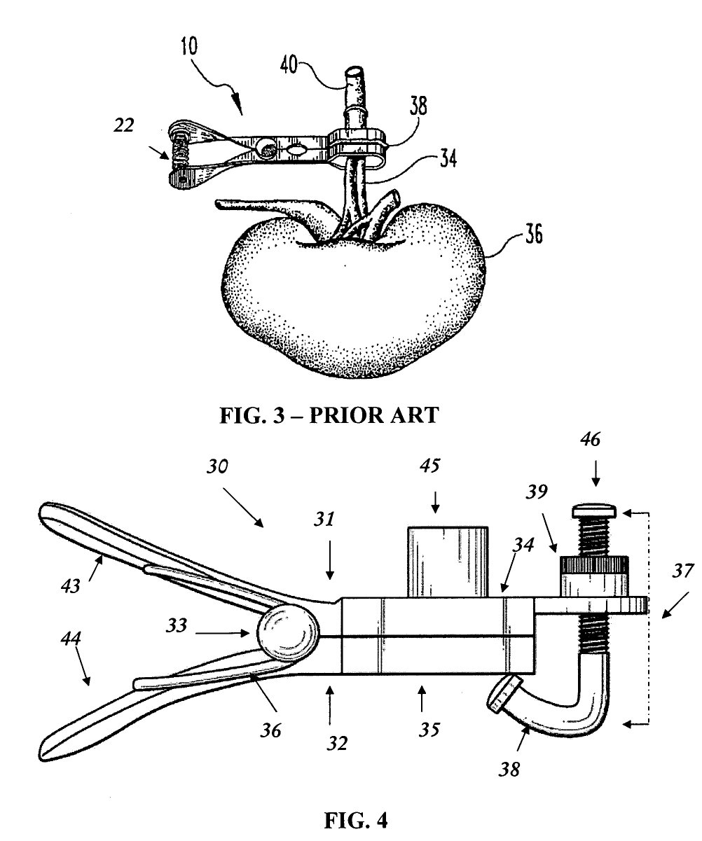

### DESCRIPTION OF THE PREFERRED EMBODIMENT

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

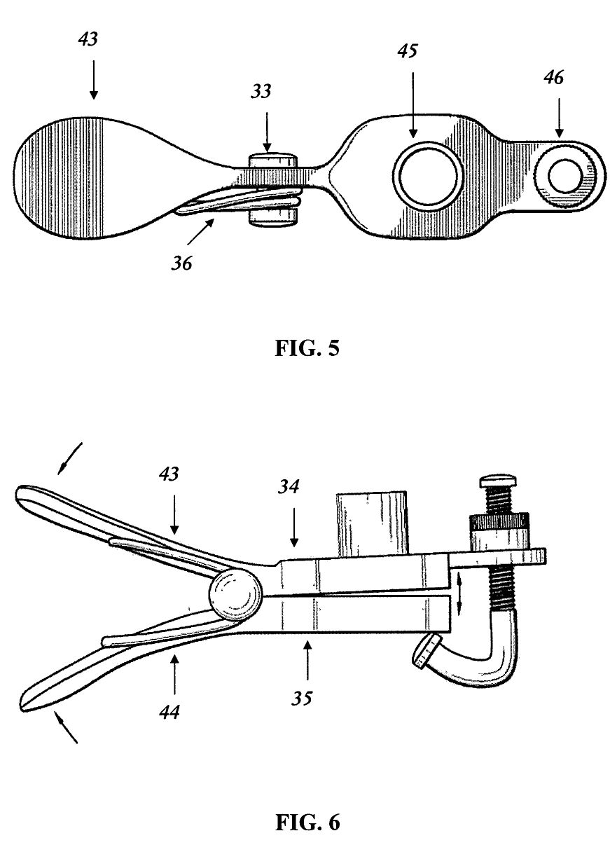

With respect to FIG. 4, the renal perfusion clamp 30 of the present invention incorporates a novel hinge design that allows the clamp to be operated with one hand and at the same time opened to approximately 60 degrees. Such a wide opening angle by means of one hand gives greater flexibility to the perfusionist when grasping the renal aortic cuff via forceps in the other hand for insertion into the clamp.

The renal perfusion clamp 30 includes two longitudinal members 31 and 32 which pivot about a hinge 33. The proximal end of longitudinal member 31 includes a handle 43. The proximal end of longitudinal member 32 includes handle 44. Handles 43 and 44 have been widened over than of prior art clamps to increase the stability of the clamp within the grip of the perfusionist as the procedure is performed.

As FIG. 4 illustrates, the distal end of longitudinal member 31 includes a clamp head 34. The distal end of longitudinal member 32 includes a clamp head 35. Movement of longitudinal members 31 and 32 toward one another forces members 31 and 32 to pivot about a hinge 33, thereby forcing clamp heads 34 and 35 of members 31 and 32 away from one another. The clamp heads 34 and 35 of the distal ends of members 31 and 32 are thus engaged in a clamping relationship unless an external compressive force is applied to members 31 and 32, as illustrated in FIG. 6. Perfusion lumen 45 extends therethrough clamp heads 34 and 35.

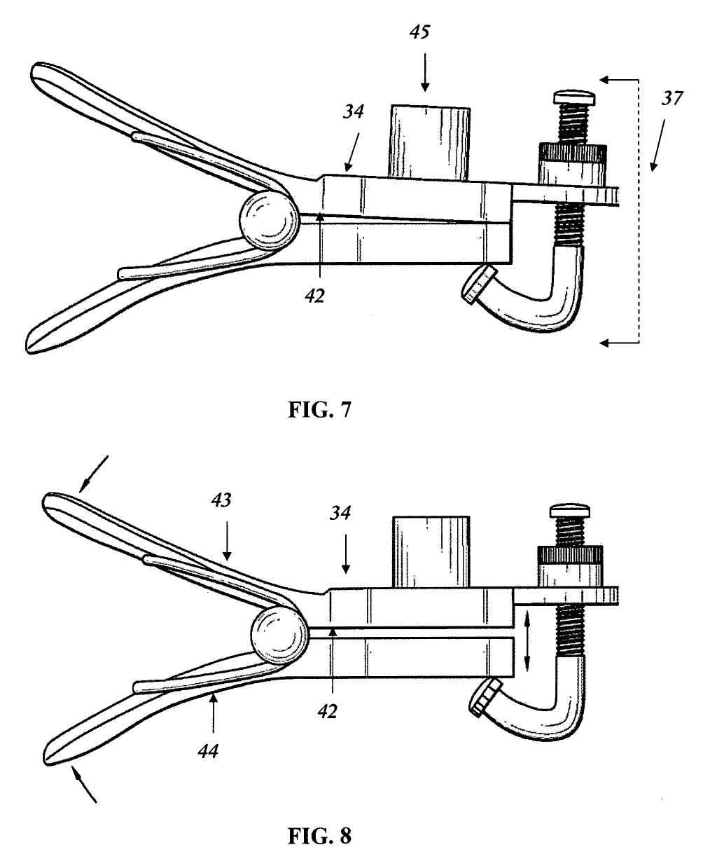

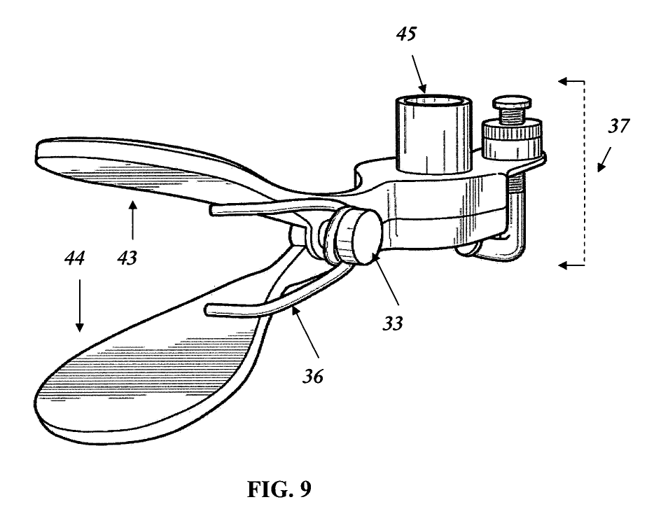

In order to further facilitate successful installation, the clamp of the present invention incorporates a spring mechanism 36, as demonstrated in FIGS. 4 through 6, to force the clamp heads to be engaged when the perfusionist has positioned the clamp correctly on an aorta patch and releases the members 31 and 32. Due to the one-handed operation of the clamp and spring mechanism 36, the perfusionist can hold the aorta patch in one hand and longitudinal members 31 and 32 of the perfusion clamp in the other hand. The perfusionist releases the clamp when the right position is reached. The clamp remains in position due to spring mechanism 36. This is illustrated in FIG. 7. Another view of the spring mechanism and hinge is set forth in FIG. 9. In order to complete an effective seal, the clamp 30 of the present invention provides for an adjustable clamping force in the form of a J-hook to the clamp's distal point, thereby giving greater clamping control to the perfusionist.

Having correctly positioned the clamp on the aorta patch, the perfusionist may now use both hands to operate the J-hook adjustable clamping force mechanism 37 without concern that the clamp's position on the aorta patch will shift incorrectly. The J-hook adjustable clamping force mechanism, illustrated in FIGS. 4, 6, 7, 8, and 9, consists of an adjustable clamping J-hook 38, a welded top cap 39, and a threaded screw 46. Welding the top cap 39 ensures that the J-hook does not get lost or separated from the clamp during the perfusion procedure.

The variable thickness of the aortic patch of a patient may extend to an approximate size of 1.0 millimeters. In the preferred embodiment of the invention, the fulcrum 42 of the clamp 30 is offset to create a gap of about 0.45 to 0.55 millimeters to provide more even sealing of the aortic patch. This corresponds to an approximate 1.0 millimeter thickness of the aortic patch. FIGS. 7 and 8 illustrate the open and closed positions, respectively, of the clamp 30 with the offset fulcrum gap placement.

The ability of the clamp to be opened to 60 degrees for ease of arterial tissue insertion makes the clamp 30 of the present invention much easier to use than prior art clamps. Moreover, the adjustable clamping J-hook 38, welded top cap 39, and threaded screw 46 provide positive clamping force between the clamp 30, thereby giving complete clamping control to the perfusionist.

The cannula length is reduced by 50% as compared to the length of prior art clamps. The length is reduced without an increase in risk of perfusion pump silicone tubing slipping off by means of increasing outside diameter of the cannula from 7 mm to 8 mm with two beneficial results. First, a more compact clamp 30 allows donor kidney to not be squeezed against opposite inner wall of perfusion pump cassette and so to not impair optimal kidney function. Secondly, the corresponding increase from 5 mm to 6 mm of inner diameter of the cannula translates to a 44% increase in theoretical flow of perfusate to kidney at prior pump pressure setting, or the same desired flow with a reduction in pump pressure. Thus, the same amount of perfusate may flow into the donor kidney at a lower pressure or an increase amount of perfusate may be allowed to flow into the donor kidney at the same pressure. The perfusionist has the option to choose either method of perfusate flow control.

Varying sizes and thicknesses of the aortic patch and varying amounts of calcification therein may call for adjustaments in the size of clamp heads. In order the minimize the number of eliminate the disadvantages associated with the repeated removal and attachments of interchangeable clamp heads, as found in the prior art, the perfusion clamp 30 present invention may be manufactured plurality of sizes each with different size clamp heads (e.g. 6×6 mm, 10×15 mm, 10×25 mm and 10×30 mm).

It will therefore be appreciated by those skilled in the art that the clamp 30 of the present invention represents a substantial improvement over the prior art.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. For example, the perfusion clamp of the present invention may be used in conjunction with perfusion of any body organ, and is not restricted to renal perfusion.

What is claimed is:

1. A perfusion clamp comprising:
(a) a first longitudinal arm having a distal end and having a first clamp head at the distal end;
(b) a second longitudinal arm having a distal end and having a second clamp head at the distal end, said second clamp head including a perfusion lumen extending therethrough;
(c) a hinge, wherein a first end of the hinge is coupled to a midsection of the first arm and a second end of the hinge is coupled to a midsection of the second arm, and wherein at least one of the first and second ends pivots at its coupling, said hinge encompassed by a spring mechanism with arms that extend between the longitudinal arms in order to bias the handles apart;
(d) said first and second longitudinal arms being configured to create an offset fulcrum gap located between the first and second arms and substantially adjacent to the hinge; and
(e) a J-hook adjustable clamping mechanism coupled to the first and second arms and operative to releasably maintain the first and second clamp heads in clamping engagement with adjustable force.

2. The perfusion clamp of claim 1, wherein the perfusion lumen contains a cannula, and wherein said lumen and cannula each have an outside diameter of approximately seven millimeters.

3. The perfusion clamp of claim 1, wherein the perfusion lumen contains a cannula, and wherein said lumen and cannula each have an inside diameter of approximately five millimeters.

4. The perfusion clamp of claim 1, wherein the first and second clamp heads may be biased apart to an opening of 45 to 60 degrees.

5. The perfusion clamp of claim 1, wherein the hinge is offset to approximately 0.5 millimeters

6. The perfusion clamp of claim 1, in which the J-hook adjustable control mechanism consists of an adjustable clamping J-hook, a welded top cap, and a threaded screw.

7. The perfusion clamp of claim 1, in which the J-hook adjustable control mechanism may also consist of a welded bottom cap.

8. A perfusion clamp comprising:
(a) a first longitudinal arm having a distal end and having a first clamp head at the distal end;
(b) a second longitudinal arm having a distal end and having a second clamp head at the distal end, said second clamp head including a perfusion lumen extending therethrough;
(c) a hinge, wherein a first end of the hinge is coupled to a midsection of the first arm and a second end of the hinge is coupled to a midsection of the second arm, and wherein at least one of the first and second ends pivots at its coupling, said hinge encompassed by a spring mechanism with arms that extend between the longitudinal arms in order to bias the handles apart; and
(d) a J-hook adjustable clamping mechanism coupled to the first and second arms and operative to releasably maintain the first and second clamp heads in clamping engagement with adjustable force.

9. The perfusion clamp of claim 8, wherein the perfusion lumen contains a cannula, and wherein said lumen and cannula each have an outside diameter of approximately seven millimeters.

10. The perfusion clamp of claim 8, wherein the perfusion lumen contains a cannula, and wherein said lumen and cannula each have an inside diameter of approximately five millimeters.

11. The perfusion clamp of claim 8, wherein the cross member is a hinge.

12. The perfusion clamp of claim 8, wherein the first and second clamp heads may be biased apart to an opening of substantially 45 to 60 degrees.

13. The perfusion clamp of claim 8, hinge is offset to substantially 0.5 millimeters.

14. The perfusion clamp of claim 8, in which the J-hook adjustable control mechanism consists of an adjustable clamping J-hook, a welded top cap, and a threaded screw.

15. The perfusion clamp of claim 8, in which the J-hook adjustable control mechanism may also consist of a welded bottom cap.
